(19) United States
(12) Patent Application Publication
(10) Pub. No.: US 2008/0154186 A1
(43) Pub. Date: Jun. 26, 2008

(54) MULTIPLE LUMEN CATHETER WITH PROXIMAL PORT

(75) Inventors: William M. Appling, Granville, NY (US); Theodore J. Beyer, Queensbury, NY (US); Carol L. Lancette, Hudson Falls, NY (US)

Correspondence Address: ANGIODYNAMICS, INC., 603 QUEENSBURY AVENUE, QUEENSBURY, NY, 12804, US

(73) Assignee: ANGIODYNAMICS, INC., Queensbury, NY (US)

(21) Appl. No.: 12/046,926

(22) Filed: Mar. 12, 2008

Related U.S. Application Data
(63) Continuation-in-part of application No. 11/557,369, filed on Nov. 7, 2006.

(51) Int. Cl. A61M 25/14

(52) U.S. Cl. 604/43

(57) ABSTRACT

A vascular access catheter is disclosed that has a catheter shaft distal tip with an angled edge positioned at an acute angle relative to a longitudinal axis of the catheter shaft. A first, second, and third lumen extend longitudinally through the entire catheter shaft. The third lumen is configured for receiving a guidewire. The first lumen has an aperture located in the angled edge of the catheter next to the distal tip and communicates with the first lumen. The second lumen has an aperture that is positioned in the outer surface of the catheter shaft that is in communication with the second lumen, and is spaced proximally from the first lumen aperture. A port is positioned in the outer surface of the catheter shaft that is in communication with the third lumen and the outer surface of the catheter shaft, and is spaced proximally from the second lumen aperture.

### CROSS REFERENCE TO RELATED APPLICATIONS

This application is a continuation-in-part application of U.S. application Ser. No. 11/557,369, filed Nov. 7, 2006, and is incorporated herein by reference.

### BACKGROUND

The present invention pertains to the field of medical devices. More particularly, the present invention relates to blood treatment catheters and a method of using such catheters.

Hemodialysis is a method for removing waste products such as potassium and urea from the blood, such as in the case of renal failure. During hemodialysis, waste products that have accumulated in the blood because of kidney failure are transferred via mass transfer from the blood across a semi permeable dialysis membrane to a balanced salt solution. The efficiency of a hemodialysis procedure depends on the amount of blood brought into contact with the dialysis membrane. A flow of 250 milliliters of blood per minute under a pressure gradient of 100 millimeters of mercury is considered a minimum requirement for adequate dialysis. Over the past several years, flow rates between 350 milliliters per minute and 400 milliliters per minute have become common.

The long hours and the frequency of the dialysis treatment in patients with renal failure require reliable, continued access to the venous system for blood exchange. Long-term venous access mechanisms commonly used for hemodialysis treatment include vascular access ports, dialysis grafts, and hemodialysis catheters. One type of blood treatment catheter that is well-known in the art is a triple-lumen hemodialysis catheter. These catheters are designed to provide long-term access to the venous system for dialysis. Triple lumen hemodialysis catheters typically have an inflow lumen for withdrawing blood to be treated from a blood vessel and an outflow lumen for returning cleansed blood to the vessel. The distal segment of the catheter is preferably positioned at the junction of the superior vena cava and right atrium to obtain a blood flow of sufficient volume to accommodate dialysis treatment requirements. This allows blood to be simultaneously withdrawn from one lumen, to flow into the dialysis circuit, and be returned via the other lumen. Hemodialysis catheters may have an additional lumen that may be used for guidewire insertion, administration and withdrawal of fluids such as antibiotics, chemotherapeutics or other drugs, blood sampling, hydration, parenteral nutrition, or injection of contrast media required for imaging procedures.

To optimize blood flow rates during dialysis and reduce treatment times, triple lumen catheters have been designed to maximize the cross-sectional lumen area of the inflow and outflow lumens. It is well known in the art that blood flow rates are negatively impacted if the cross-sectional area of the lumens does not remain essentially consistent and as large as possible throughout the entire length of the catheter from the proximal portion of the catheter to the distal portion of the catheter. Catheters with large, consistent luminal space typically have exit ports with blunt or flat-faced open tips, so as not to compromise the luminal area. Typically the exit port at the distal end of the catheter is cut at a 90 degree angle to the axis of the catheter.

Blunt, open ended catheters maintain optimal flow rates, but they are difficult to insert into the patient because of their blunt leading ends. An introducer sheath will often be used to facilitate insertion. The introducer sheath has a dilating tip that is easily advanced through a tissue track and into the vessel. The sheath has a large lumen into which the blunt-tipped catheter is inserted and advanced into the vessel. Although an introducer sheath may facilitate catheter placement, use of a sheath has several disadvantages. A sheath increases the risk of air embolism due to the presence of air gaps between the sheath and catheter. In addition, procedures that use an introducer sheath result in an enlarged insertion tissue track due to the larger diameter of the sheath relative to the catheter. The use of a sheath also increases procedure time and costs.

A guidewire insertion technique is therefore often the preferred insertion technique for dialysis catheter placement. A guidewire is a thin, flexible wire that is usually made of stainless steel and has an atraumatic tip. A guidewire is typically inserted into a lumen of a triple lumen catheter, and then the catheter is advanced over the guidewire through the tissue track and into the vessel. The guidewire also provides additional stiffness or reinforcement to the catheter, to minimize kinking or accordianing of the catheter shaft as it is advanced through the tissue track and into the vessel.

If a guidewire is used for insertion of a blunt-end catheter with a large distal end opening, excess space will exist between the outer diameter of the guidewire and the inner diameter of the catheter lumen. A close fit between the lumen and the inserted guidewire is not dimensionally possible, thus leaving an annular gap between the guidewire and the distal opening of the catheter lumen. The excess annular space causes the leading distal edge of the catheter to accordion proximally over the guidewire during insertion, resulting in difficulties in advancing the catheter into the vessel. The distal portion of the catheter may grab or snare tissue as the practitioner attempts to advance the catheter into and through the vessel. This can increase procedure time, prevent the practitioner from reaching the intended target site within a patient's vessel, or potentially cause other complications.

To overcome insertion difficulties common with inserting blunt tipped catheters, dialysis catheters have been designed with conical tapered distal portions that are narrower compared to the proximal portion of the catheter. The conical tip acts as a dilator to facilitate advancement of the catheter through the tissue track and into the vessel. These conical tip designs may include a guidewire lumen that exits from the distal tip of the catheter through a guidewire opening of reduced diameter, conventionally 0.037 inches.

While conical, tapered tip designs address the problems associated with inserting blunt tip full lumen distal end designs, they are disadvantageous in that they do not allow for optimum flow rates due to the reduced lumen diameter at the distal tip. To overcome reduced flow rates, conical, tapered tip catheters have been designed with distal side facing ports or apertures cut through the catheter sidewall. The ports are located proximal to the conical tapered section and accordingly provide an exit channel from the lumen at a location where the cross-sectional area of the lumen has not been reduced.

Using side holes or apertures eliminates the problems of reduced flow rates, but side-facing apertures are more likely to occlude than distally facing apertures. Those side holes located adjacent to the vessel wall are more likely to become blocked by the vessel wall, and are thus prone to clot-formation. In addition, the presence of side holes compromises the effectiveness of a fluid lock. A fluid lock, as known in the art, is used to prevent clot formation within the catheter between dialysis sessions. Typically, a heparin-saline fluid solution is infused into the full length of the catheter lumens. The fluid lock will only be effective up to the first proximal side hole, where the fluid will exit from the catheter and be replaced by blood. In the absence of the heparin-saline fluid solution, a portion of the lumen distal of the first side hole will become occluded by clot formation, complicating future dialysis sessions.

Another common complication of dialysis catheters is occlusion of the inflow and outflow apertures due to contact between the catheter and the vessel wall at the location of the apertures. During dialysis, negative pressure is generated within the inflow lumen in order to draw blood from the vessel through the lumen and into the dialysis machine. The suction created by the negative pressure may cause the catheter to move away from the center of the vessel and into contact with the vessel wall. The vessel wall essentially blocks the aperture, preventing further blood from being drawn into the inflow lumen. Although not as common, the outflow apertures may also come to rest up against the vessel wall, resulting in occlusion. Occlusion can result in treatment delay, surgical replacement, patient discomfort, and increased cost of care.

Other potential complications related to the placement of triple lumen hemodialysis catheters may include infection, reduced or uneven blood flow, thrombosis, thrombophlebitis, or fibrin sheath formation on the catheter shaft. Fibrin sheaths can form on an implanted catheter just a few days after implantation. One possible theory of fibrin sheath growth is that the fibrin sheath begins growing from the vein entry point, and progresses toward the catheter tip. Despite fibrin sheath build up, infused fluids may enter the blood circulation, but when negative pressure is applied, the fibrin sheath can be drawn into the catheter, occluding its tip, thereby preventing aspiration. Complete encasement of the catheter tip in a fibrin sheath may cause persistent withdrawal occlusion. This can lead to extravasation of fluid where fluid enters the catheter to flow into the fibrin sheath, backtracks along the outside of the catheter, and exits out of the venous entry point and into the tissue.

The growth of a fibrin sheath along a catheter shaft can prevent high flow rates, adversely affect blood sampling and infusion of chemotherapeutic drugs, and provide an environment in which bacteria can grow, which may result in infections. This is problematic when using triple lumen hemodialysis catheters because in patients who need prolonged intravenous regimens and have poor peripheral venous access, hemodialysis catheters are often the only means available for the delivery of necessary treatment. Therefore, such hemodialysis catheters should be configured to remain patent so that drugs and other fluids can be effectively delivered to a patient's vasculature and to break up any fibrin sheath growth.

Fibrin sheaths may be removed by mechanical disruption or stripping with a guidewire or loop snare, or by replacing the catheter. Mechanical disruption can help prevent the need to replace the catheter, and thereby eliminate disruption to the patient. However, mechanical disruption may not be effective because the fibrin sheath may not be radiographically detectable before mechanical disruption is attempted. Mechanical disruption may also cause trauma to the patient or damage to the catheter. Replacing the catheter is also an option, but this can cause increased trauma to the patient, increased procedure time and costs, increased risks of pulmonary emboli, and may require numerous attempts before removal is successful. Thus, both mechanical disruption and catheter replacement may adversely affect a patient's dialysis schedule, cause patient discomfort, and loss of the original access site.

The infusion of drugs, such as urokinase, may be used to break up fibrin sheath formation. This treatment option is a more cost-effective, time-saving method that helps preserve an implanted catheter, prevents the patient from experiencing additional trauma, and preserves the primary access site. Other drugs, such as streptokinase, or tissue plasminogen activator (t-PA), may also be used. These drugs activate the enzyme plasminogen, which triggers fibrinolysis, which causes the fibrin sheath to dissolve.

Given the high likelihood of fibrin sheath formation on implanted hemodialysis catheters, it is desirable to use hemodialysis catheters that, in addition to optimal blood flow rates, allow for the infusion of thrombolytic drugs, particularly in situations where patients require the administration of multiple fluids. Triple lumen catheters have been developed in which distal apertures that exit the catheter shaft and are used to deliver drugs, such as urokinase or anti-thrombotic agents, to the surrounding tissue. An “overfill technique” is sometimes used to deliver drugs to fibrin sheaths in these catheters. This over-filling technique is time-consuming and expensive because each lumen has to be separately infused with urokinase, so that at least some of the urokinase will come into contact with the fibrin sheath to dissolve the fibrin sheath. This delivery method may not adequately break up fibrin sheaths, however, because the distal lumen apertures may become occluded, as described above, and thus, the drug may not reach the fibrin sheath. A fibrin sheath located at the tip of a catheter may not be dissolved if it is not contacted by the drug or sufficient concentrations of the drug. A drug injected through the distal most aperture of the catheter is not likely to reach a fibrin sheath because the drug will be delivered in the direction of the blood flow, instead of flowing backward along the catheter shaft where the fibrin sheath is located.

Triple lumen catheters have been developed that have ports or apertures in a third lumen for the delivery of drugs or medications, but such ports or apertures may not be optimally located near a potential fibrin sheath, or they may not be adaptable for the infusion of drugs and insertion of a guidewire. Alternatively, catheters have been proposed with drug infusion side ports that exit through other lumens of the catheter, but such ports may be located in positions along the catheter such that optimal flow rates are compromised. These ports or openings in the catheter may become easily blocked by the vessel wall or may cause fibrin to collect in one location along the catheter, blocking the apertures of the catheter with fibrin, which could possibly spread up into the lumens of the catheter, or may cause weak areas or kinking points along the catheter shaft.

Thus, there exists a need in the art for a triple lumen hemodialysis catheter that has a dilating distal tip that is not reduced in cross-sectional area relative to the rest of the lumen. Such a catheter can be designed to prevent occlusion of the blood lumen apertures by having a distal end shape that creates a barrier between the blood lumens and vessel wall. Such a lumen can maintain consistent and optimal blood flow rates throughout the entire length of the catheter without the need for side hole ports in the inflow or outflow lumens of the catheter. The catheter can have one lumen capable of receiving a guidewire that can provide enhanced guidewire tracking along the entire length of the catheter, thereby eliminating the need for an introducer sheath. The third lumen can also be capable of effectively infusing chemotherapeutic or antithrombotic drugs into or in the general vicinity of any fibrin sheath buildup on the catheter shaft, such that the fibrin sheath may be quickly dissolved.

A hemodialysis catheter has not yet been proposed that solves all of the above-mentioned problems. A triple lumen hemodialysis catheter is provided herein that has at least two lumens, each with at least one aperture, and a distal portion that has one lumen with a substantially open tapered face distal end portion with a distal tip and consistent cross-sectional area compared to the rest of the lumen of the catheter, which allows for maximum blood flow. In one aspect, the catheter can also have a third lumen located adjacent the distal tip that is capable of receiving a guidewire and which has at least one proximal port through which drugs, such as urokinase, can be infused to effectively break up fibrin sheath buildup or other occlusive material along the catheter shaft. In this aspect, the guidewire aperture and the tapered face of the distal end portion facilitate insertion, without the use of an introducer sheath. In a further aspect, the luminal cross-sectional area can be maintained for the substantial length of the catheter. The catheter may optionally include a curved or bent distal end shape to prevent contact between the lumen apertures and the vessel wall.

Accordingly, provided herein, in one aspect, is a hemodialysis catheter that has three lumens and a tapered open-faced distal end portion that provides for optimal blood flow rates by maintaining a uniform cross-sectional area throughout the lumen. In this aspect, it is contemplated that the need for attachments or additional steps would be eliminated, thereby minimizing procedure time and improving patient treatment outcomes.

A further purpose is to provide a catheter that maintains the cross-sectional area of the blood lumen of the catheter without increasing the outer diameter of the catheter.

A further purpose is to provide a catheter that minimizes occlusion of the lumen apertures of the catheter by providing a substantially curved distal portion that abuts against the vessel wall while the catheter is deployed in a vessel. The abutting substantially curved distal portion acts to guard one of the lumen apertures of the catheter from being occluded, which in turn, maintains maximum blood flow.

A further purpose is to provide a catheter that has a distal portion that allows for increased ease of insertion of the catheter into a vessel. The insertion is facilitated by straightening the distal portion of the catheter from a substantially curved to a straight configuration, which causes less resistance upon insertion. The distal portion of the catheter is more flexible, compared to the rest of the catheter, which helps to facilitate straightening of the distal portion. The flexibility of the distal portion of the catheter allows the distal portion to return to its original configuration after the guidewire is removed.

It is yet a further purpose to provide a catheter that maximizes flow rates without requiring side hole ports in the inflow or outflow lumens of the catheter.

It is yet another purpose to provide a non-conical distal end portion catheter that may be placed without the use of an introducer sheath.

A further purpose is to provide a catheter that is capable of receiving a guidewire in a third lumen that extends substantially entirely through the catheter to the proximal end of the catheter that is designed for optimal guidewire tracking without requiring the use of an introducer sheath and which may also be used for injections or infusion of drug treatments, such as urokinase, to break up a fibrin sheath or similar occlusive material.

A further purpose is to provide a catheter with a third lumen that has at least one port that is strategically placed along the catheter shaft such that it exits the third guidewire lumen proximally of the inflow lumen aperture of the catheter, such that when drugs are infused through this exit port, such drugs may exit directly into or as near to a fibrin sheath or other occlusive material as possible, so as to dissolve the fibrin sheath during hemodialysis, thereby overcoming the disadvantages of other triple lumen hemodialysis catheters.

Various other purposes and embodiments of the present invention will become apparent to those skilled in the art as more detailed description is set forth below. Without limiting the scope of the invention, a brief summary of some of the claimed embodiments of the invention is set forth below. Additional details of the summarized embodiments of the invention and/or additional embodiments of the invention may be found in the Detailed Description of the Invention.

### SUMMARY

In one embodiment, a vascular access catheter is disclosed that has a catheter shaft with a distal portion that has a distal end portion with a distal tip having an angled, leading edge that is positioned at an acute angle from the distal tip relative to a longitudinal axis of the catheter shaft. A first, second, and third lumen extend longitudinally through the catheter shaft. The third lumen is configured for receiving a guidewire. The first lumen has an aperture located in the angled edge distal end portion of the catheter next to the distal tip and communicates with the first lumen. The second lumen has an aperture that is positioned in the outer surface of the catheter shaft that is in communication with the second lumen, and is spaced proximally from the first lumen aperture. The catheter shaft has a port positioned in the catheter shaft that is in communication with the third lumen and the outer surface of the catheter shaft, and is spaced proximally from the second lumen aperture.

In another embodiment the vascular access catheter has a catheter shaft that has a distal end portion having a distal tip without an angled edge. A first, second, and third lumen extend longitudinally through the catheter shaft. The first and second lumens are separated by and share a common internal septum. The first lumen has a smaller transverse cross-sectional area than the second lumen. The third lumen is configured for receiving a guidewire. The first lumen has an aperture located in the distal end portion of the catheter and communicates with the first lumen. The second lumen has an aperture that is positioned in the outer surface of the catheter shaft, is in communication with the second lumen, and is spaced proximally from the first lumen aperture. The catheter shaft has a port positioned in the catheter shaft that is in communication with the third lumen and the outer surface of the catheter shaft, and is spaced proximally from the second lumen aperture at an acute angle from the longitudinal axis of the catheter shaft.

In a further aspect, a method of using the catheter is also provided that involves inserting the catheter into a patient body. In yet a further aspect, a method is provided that involves infusing drugs or other chemotherapeutic agents into the third lumen and through the proximal port into a patient body in order to dissolve occlusive material along the catheter shaft, such as that found in fibrin sheaths.

### BRIEF DESCRIPTION OF THE SEVERAL VIEWS OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1A is a plan view of a vascular access catheter with a substantially curved distal portion.

FIG. 1B illustrates cross-sectional views of the catheter shaft of the catheter of FIG. 1A, along lines A-A and B-B, respectively.

FIG. 1C is an exploded view of the distal portion of the catheter shaft of FIG. 1A.

FIG. 2A is an enlarged partial cross-sectional view of the distal end of the vascular access catheter of FIG. 1A, with a portion of a guidewire inserted into the third lumen.

FIG. 2B illustrates two different cross-sectional views of the catheter of FIG. 2A at the catheter shaft, with a portion of a guidewire inserted into the third lumen, along lines D-D and C-C, respectively.

FIG. 3A is an enlarged partial cross-sectional view of the catheter shaft of FIG. 2A, with a portion of a guidewire inserted into the third lumen.

FIG. 3B is an enlarged partial cross-sectional view of an additional embodiment of the catheter shaft of FIG. 2A.

FIG. 3C is an enlarged partial cross-sectional view of the embodiment of the catheter shaft of FIG. 3B, with a portion of a guidewire inserted into the third lumen.

FIG. 4A illustrates a partial bottom view of the distal portion of the catheter shaft of FIG. 1A.

FIG. 4B illustrates a partial bottom view of the distal portion of the catheter shaft of FIG. 1A with a portion of a guidewire inserted into the third lumen.

FIG. 4C illustrates a partial bottom view of an additional embodiment of the distal portion of the catheter shaft of FIG. 1A.

FIG. 5A illustrates a plan view of an additional embodiment of the vascular access catheter of the present invention, with a port in the third lumen of the catheter shaft.

FIG. 5B illustrates a top plan view of the catheter of FIG. 5A.

FIG. 5C illustrates three different cross-sectional views of the catheter of FIG. 5A at the catheter shaft, along lines A-A, B-B, and C-C, respectively.

FIG. 6A illustrates an enlarged partial cross-sectional view of an additional embodiment of the distal end of the vascular access catheter of FIG. 5A.

FIG. 6B illustrates a top plan view of the distal end of the catheter of FIG. 6A.

FIG. 7 is a perspective view of the distal end of the catheter of Figure FIGS. 6A and 6B with a portion of a guidewire inserted into the third lumen.

### DETAILED DESCRIPTION

The following detailed description should be read with reference to the drawings, in which like elements in different drawings are identically numbered. The drawings, which are not necessarily to scale, depict selected preferred embodiments and are not intended to limit the scope of the invention. The detailed description illustrates by way of example, not by way of limitation, the principles of the invention.

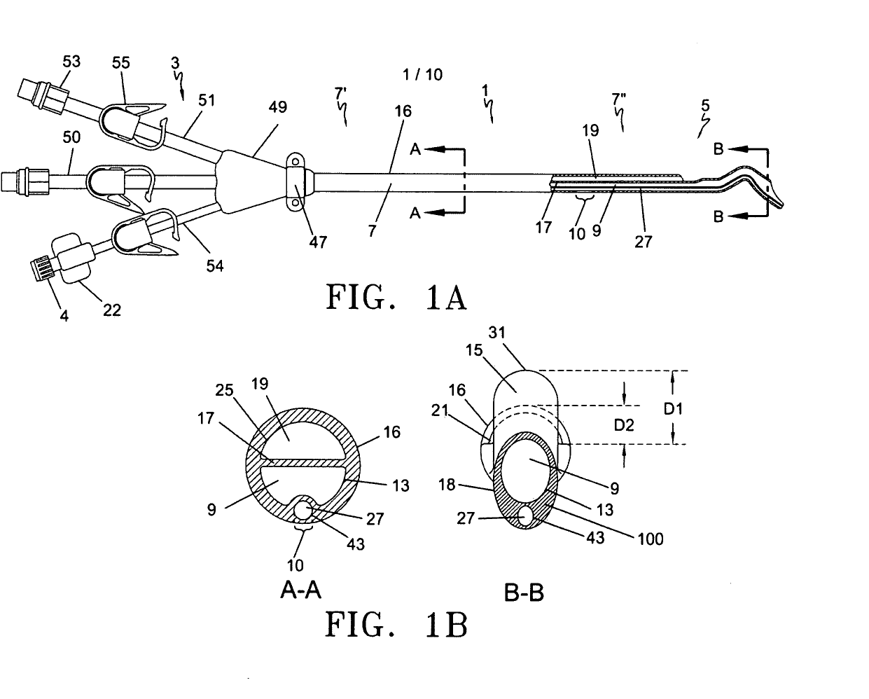

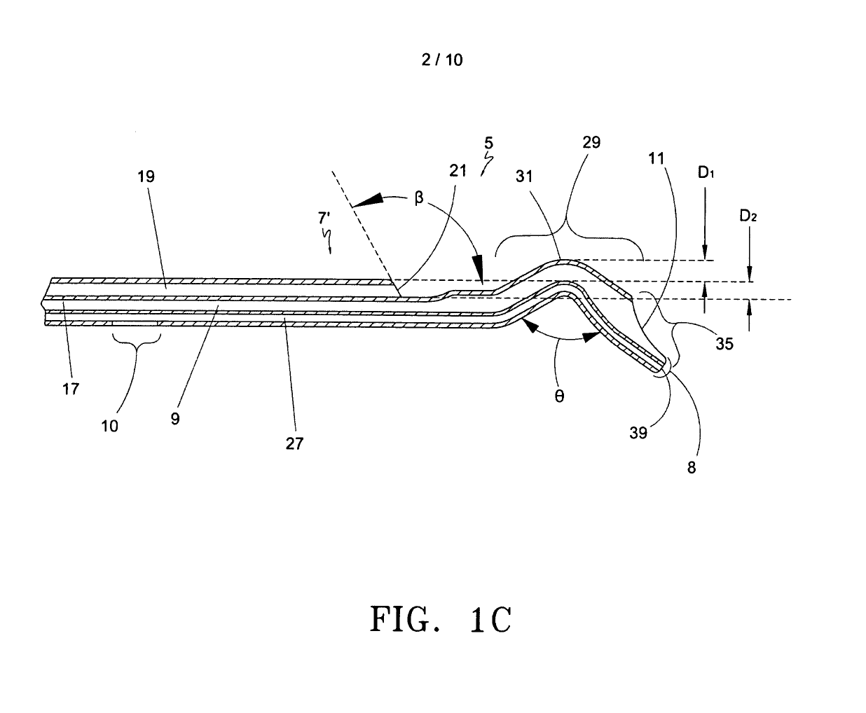

In various embodiments, and referring to FIGS. 1-7, presented herein is an exemplary vascular access catheter, such as a hemodialysis catheter, a method of inserting the catheter in the body of a patient, a method of infusing an infusate, such as drugs or other agents, into the third lumen of the catheter and into a patient body, and a method of removing occlusive material from a catheter shaft. FIG. 1A illustrates one embodiment of the hemodialysis catheter. In one aspect, the unitary catheter 1 has a proximal portion 3 and a distal portion 5. In this exemplary aspect, at least a portion of the distal portion 5 of the catheter 1 is substantially curved. It is contemplated that the at least a portion of the distal portion 5 of catheter shaft 7 may have any suitable curved shape configuration, including, but not limited to a curved, bent or semi-helical shape. Alternatively, the catheter 1 may have a distal portion 5 which may be substantially straight. In another aspect, the proximal portion 3 of the catheter 1 can be comprised of a bifurcate 49, a suture ring 47 coaxially arranged around the distal portion of the bifurcate 49, extension tubes 50, 51, 54, extension tube clamps 55, catheter hub connectors 53 for connection to a dialysis machine. In this aspect, at least a portion of the distal portion 5 of the catheter is formed by a catheter shaft 7 that extends from the bifurcate 49 to the distal tip 8 at the distal portion 5 of the catheter 1. Optionally, an infusion port 4 with an identification tag 22 may be placed on the third extension tube 54 for the infusion of drugs or chemotherapeutic agents.

In one aspect, the catheter shaft 7 can be comprised of an outer wall 16 and at least a first lumen 9 and a second lumen 19 extending longitudinally through substantially the entire length of the catheter shaft 7. Lumen 19 is in fluid communication with extension tube 51, and lumen 9 is in fluid communication with extension tube 50. Both extension tubes 50, 51 communicate through bifurcate 49. It is contemplated that any of extension tubes 50, 51, or 54 may have an identification (ID) tag denoting the flow rate and/or volume of fluid to be injected into the catheter. In one example, blood can be withdrawn from the vessel of the patient into lumen 19 where it is passed through the extension tube 51 and into the dialysis machine. Blood can be returned to the patient through extension tube 50 into lumen 9, which exits through the distal aperture 11 into the vessel of the patient.

In one example, the outer diameter of the catheter 1 is approximately 0.203 inches, although, as one skilled in the art will appreciate, other diameter catheters are within the scope of this invention. In another example, and not meant to be limiting, the usable length of the catheter shaft 7 can be between approximately 20 cm to 55 cm, depending on the patient's anatomy and physician preference. In one aspect, the catheter shaft 7 may have a usable length that is between approximately 32 and 36 cm. The term “useable length”, as defined herein, means the length from the distal edge of a cuff (not shown) of the catheter to the distal tip 8 of the catheter. A catheter cuff is typically positioned along the length of the catheter to stabilize the catheter position when implanted in tissue. The cuff may also prevent the ingrowth of tissue, and may prevent infectious agents from migrating along the length of the catheter into a patient's body. The catheter 1 may also be, but is not limited to, a 15.5 Fr catheter, although, as one skilled in the art will appreciate, any suitable size catheter may be used. In one aspect, the catheter 1 is a unitary catheter composed of carbothane, but any suitable material may be used, such as, but not limited to, polyurethane or silicone. In another aspect, the catheter 1 may also contain a radiopaque material to enhance visibility under fluoroscopy.

At least a portion of the catheter shaft 7 forms the distal portion 5 of the catheter 1. In a further aspect, the catheter shaft 7 can be configured such that the shaft is preferably softer and more flexible at its distal portion 7″ than its proximal portion 7′. In one example, and not meant to be limiting, the distal portion 7″ can have a reduced diameter and be formed with less material, compared to the proximal portion 7′ of the catheter shaft 7, such that the distal portion 7″ is relatively more flexible than the proximal portion 7′. The increased relative flexibility of the distal portion 7″ allows the distal portion 5 of the catheter to be more easily advanced through the vessel. The catheter shaft 7 may optionally be comprised of materials of different durometers to produce a shaft 7 with enhanced flexibility at the distal portion 5. In various other aspects, the catheter shaft 7 can be configured to be stiffer at the proximal portion 7′ outside of the patient's body for durability and more flexible at the distal portion 7″ to facilitate insertion of the catheter 1 and to provide a catheter 1 with an atraumatic tip, when placed within a vessel of the patient. Although the embodiments of the catheter 1 described herein do not have additional side ports or apertures in the first and second lumens 9 and 19, respectively, it is contemplated that in other embodiments of the catheter 1, the catheter shaft 7 may have side ports or apertures defined in the exterior of the catheter shaft 7 and in fluid communication with the first and second lumens, respectively.

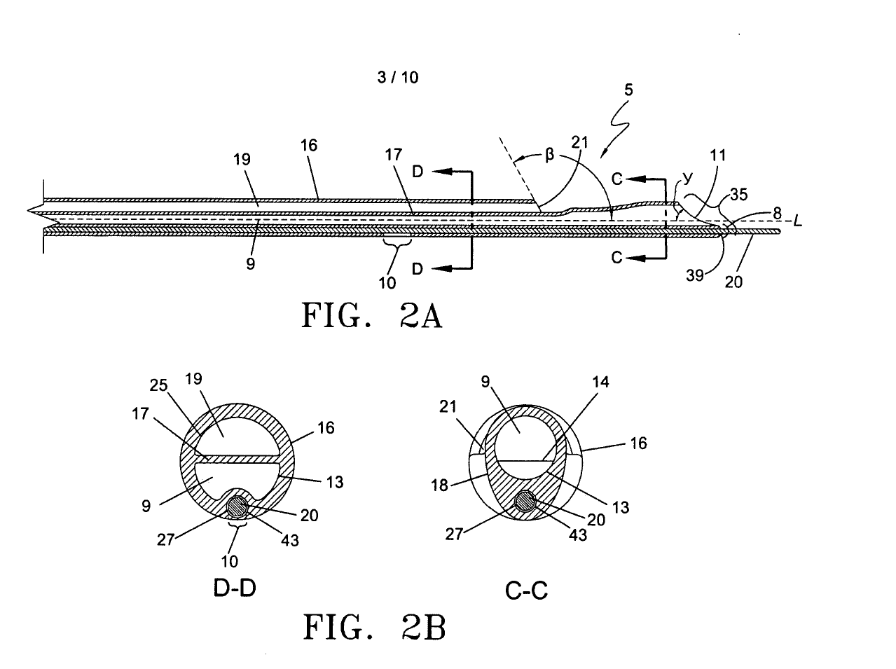

In an additional aspect, the catheter 1 can have an inflow lumen 19 that is in fluid communication with an inflow aperture 21 that is defined in the exterior surface of the catheter 7 in the distal portion 5 of the catheter 1. The inflow lumen aperture 21 is in fluid communication with the second lumen 19 and terminates proximally of the outflow lumen aperture 11. The inflow lumen 19 can be exemplarily used for withdrawal of blood from the patient. In one exemplary aspect, and as shown in FIGS. 1C and 2A, the inflow aperture 21 can be sloped such that the cross-section of the inflow aperture 21 is positioned an angle β greater than about 90 degrees relative to a longitudinal axis L the catheter shaft 7. In another aspect, the catheter 1 has an outflow lumen 9 that can be exemplarily used for delivering cleansed blood back into the patient's circulatory system. In this example, blood exits the distal portion 5 of the catheter 1 through outflow aperture 11 that is defined in the distal end portion 35 of the catheter 1 distally of the inflow aperture 21, adjacent the distal tip 8 and in communication with the first lumen 9. In yet another aspect, the two lumens 9 and 19 have inner walls 25 and 13, respectively, and are separated along their longitudinal length by a common internal septum 17, illustrated along line A-A in FIG. 1B.

One skilled in the art will appreciate that, although designated herein as inflow and outflow lumens, dialysis may be performed by reversing the blood flow through the lumens. Hence, the terms first lumen and second lumen may also be used herein to designate the interchangeability of the outflow and inflow lumens, respectively. In one aspect, the inflow lumen 19 can have a D-shaped lumen configuration, and the outflow lumen 9 can have a partially D-shaped lumen configuration. Of course, it is contemplated that the lumens of the catheter 1 may have any suitable cross-sectional lumen shape as required for the particular use of the catheter 1.

In one aspect, the catheter 1 can have a third guidewire lumen 27 that extends proximally from a guidewire exit aperture 39 defined therein the distal portion 5 of the catheter and through the entire length of the catheter 1 from the distal tip 8 to the bifurcate 49, where the guidewire lumen 27 is fluidly joined to extension tube 54 at the proximal portion 3 of the catheter. In one example, the third lumen 27 can have a generally smaller transverse cross-sectional area than lumens 19 and 9. In this aspect, the guidewire lumen 27 is configured for slidably receiving at least a portion of a guidewire (not shown). The guidewire lumen 27 provides a guidewire track for the guidewire to facilitate insertion of the catheter 1 through tissue into the target vessel and allows for improved guidewire insertion and tracking techniques. In one exemplary aspect, and not meant to be limiting, the inner diameter of guidewire lumen 27 may be approximately 0.037 inches so as to accommodate a guidewire with an outer diameter of approximately 0.035 inches, such that it is positioned in close surrounding relationship to at least a portion of inserted guidewire 20. It is contemplated that other dimensions may be used for the third lumen 27 and the guidewire 20, provided such dimensions provide a close surrounding relationship between the third lumen 27 and at least a portion of guidewire 20. These dimensions allow the guidewire 20 to be slidably received within the lumen 27, while minimizing space between the outer diameter of the guidewire 20 and the inner diameter of the lumen 27.

This enhanced guidewire tracking prevents tissue from being snagged during advancement of the catheter 1 into a target location, and the distal end portion 35 provides a dilating function, thereby reducing trauma and tissue disruption to the vessel. The guidewire 20 and catheter 1 may therefore be easily inserted into a vessel without requiring the use of an introducer sheath. One skilled in the art will appreciate that the elimination of the introducer sheath reduces procedure time and costs and minimizes the risk of air embolism due to absence of air gaps between the sheath and the catheter 1. In this aspect, the outer diameter of the catheter 1 does not have to be increased to accommodate the lumen 27 adjacent to the outflow lumen aperture 11 at the distal most edge of the distal tip 8. This allows the effective cross-sectional area of the outflow lumen 9 to be maintained to be substantially uniform throughout the catheter 1 and provides for maximum blood flow.

It is contemplated that the guidewire lumen 27, which is fluidly connected with extension tube 54, may be used for the injection and delivery of infusates, such as, for example and without limitation, drugs, such as urokinase or other anti-thrombotic agents, fluids, such as contrast media, or for blood sampling, eliminating the need for the practitioner to place a secondary vascular access device. In another aspect, the continuous guidewire lumen 27 allows for guidewire exchange or re-insertion, if necessary, after the catheter 1 has been placed in a vessel.

In one aspect, at least one port 10 may optionally be defined in the catheter shaft 7 that is in fluid communication with the third lumen 27 and the exterior of the catheter shaft 7. In one example, the at least one port 10 is defined in the exterior surface of the catheter shaft 7 and is spaced proximally from inflow aperture 21. In this aspect, the port 10 can be positioned along the catheter shaft 7 between approximately 5 cm and 7 cm from the distal tip 8 of the catheter 1. In the embodiment illustrated in FIG. 1A, the port 10 exits the third lumen 27 along the bottom of the catheter shaft 7, i.e., in opposition to the inflow aperture 21 that is positioned on the top of the catheter shaft 7. In other aspects, the port 10 may be defined thereon the catheter shaft 7 at any desired position on the catheter shaft 7. The port 10 allows for the efficient for infusion of infusates, such as drugs, into the third lumen 27 and through the port 10 to dissolve fibrin sheaths or other occlusive material that has the tendency to form along the catheter shaft 7 after the catheter 1 has been implanted in a patient body, as described further herein.

In one aspect, the distal tip 8, outflow aperture 11, and the guidewire exit aperture 39 define a distal end portion 35 of the distal portion 5 of the catheter 1 in which at least a portion of the distal end portion 35 is tapered. The term “tapered,” as it pertains to the description herein, means that at least a portion of the distal end portion 35 has an angled edge that is not at a perpendicular angle relative to the longitudinal axis of the catheter 1, and could exemplarily include end portions 35 defined by flat, arcuate, or extended arcuate surfaces. In one aspect, the angled edge is angled proximally away from the distal tip 8 and is positioned at an acute angle γ, illustrated in FIG. 2A, relative to a longitudinal axis of the catheter shaft 7. In one aspect, the tapered distal end portion 35 is positioned at approximately 30 degrees relative to the longitudinal axis of the catheter 1. In this aspect, the tapered distal end portion 35 that extends from the proximal most edge of the outflow lumen aperture 11 to the distal most edge of the third lumen 27 is approximately 5 mm, although the length can vary based on the angle of the slope. The acute angle γ of the angled edge of at least a portion of the distal end portion 35 may between approximately 15 degrees and 75 degrees relative to the longitudinal axis of the catheter shaft 7. In another exemplary aspect, the tapered distal end portion 35 can be configured to act as a dilator to provide enhanced insertion and tracking functionality without compromising flow rates, as will be explained in greater detail below.

The distal portion 5, defined herein as the length between the distal most edge of the inflow aperture 21 and the distal most edge of the guidewire exit aperture 39, can, in one non-limiting example, be approximately 2.5 cm, and in the depicted embodiment in FIG. 1A, be substantially curved. In this example, the length between the distal most edge of the inflow aperture 21 and the proximal most edge of the outflow lumen aperture 11 is approximately 2 cm, which provides sufficient separation between the respective outflow and inflow lumens 9, 19 to minimize re-circulation of blood during dialysis. As one skilled in the art will appreciate, recirculation is a complication of dialysis in which treated blood exiting from the outflow aperture 11 is pulled back into the catheter 1 through the inflow aperture 21 and re-processed by the dialysis machine. Recirculation reduces the efficiency of the cleansing process and results in inadequate dialysis if recirculation rates are too high. By spacing the inflow aperture 21 and outflow aperture 11 sufficiently apart, the recirculation rate during treatment can be reduced to an acceptable level.

FIG. 1B illustrates the cross-sectional area of the catheter 1 of FIG. 1A taken along lines A-A and B-B. As noted above, the outflow lumen 9 and the inflow lumen 19 are separated by a common internal septum 17. In this example, the third guidewire lumen 27 is defined in at least in the region of the distal portion 5 of the catheter and has an inner wall 43. In the embodiment illustrated in FIGS. 1A and 1B, the third lumen 27 can be centered below the outflow lumen 9, such that the luminal cross sectional area of the outflow lumen 9 is not comprised. In this example, the distal aperture 39 of the third lumen 27 is defined in the distal portion 5 and is positioned distal to the outflow lumen aperture 11 at the distal most portion of the angled edge.

In a further aspect, to accommodate the guidewire lumen 27 within the partial double-D section of the catheter 1 without increasing the outer diameter of the catheter 1, the internal septum 17 can be positioned slightly off-center. This allows for the effective cross-sectional area of each lumen 19 and 9 to be substantially equalized, and aids in providing substantially equalized flow rates in both the inflow and outflow directions. In one non-limiting example, the resulting cross-sectional area of each respective lumen 19 and 9 is approximately 0.0065 inches². Thus, in this aspect, the cross-sectional luminal areas of the catheter 1 are maintained without having to increase the outer diameter of the catheter.

As illustrated in FIG. 1C, at least a portion of the distal portion 5 of the catheter 1 that is substantially curved defines a guard portion 29. In this aspect, the guard portion 29 has an apex 31 that is located at the outermost point of the guard portion 29. A portion of the guard portion 29 is spaced from the longitudinal axis of the catheter shaft 7 (shown as line “L” in FIG. 1A) a distance D1 that is equal to or greater than the distance D2 that the outer wall 16 of the inflow aperture 21 is spaced from the longitudinal axis of the catheter shaft 7. The substantially curved distal portion 5 acts to guard lumen aperture 21 of the catheter 1 from being occluded, which in turn, maintains maximum blood flow. The guard portion 29 is also defined by an inner angle θ opposite the apex 31. In various aspects, it is contemplated that when the catheter shaft 7 is in the unstressed state, the inner angle θ of at least a portion of the distal portion 5 of the catheter may be between approximately 45 degrees and 135 degrees. In another aspect, the inner angle θ can be equal to or greater than about 90 degrees, depending on the curvature of the guard portion 29. In yet another aspect, the inner angle θ can be approximately 90 degrees. Optionally, the curved distal portion 5 may have substantially straight portions on either side of the inner angle θ, or the curved distal portion 5 may be a substantially continuous series of arcuate arcs.

In a further aspect, the space between the apex 31 and the outer wall 16 of the inflow aperture 21 can be configured to function as a guard to prevent the aperture 21 from moving up against the vessel wall (not shown) and at least partially or fully occluding the inflow aperture 21. In this aspect, as described above, the height D1 of apex 31 in relation to the longitudinal axis of the catheter shaft 7 can be configured to be equal to or greater than the height D2 of the outer wall 16 of inflow aperture 21 in relation to the longitudinal axis of the catheter shaft 7, which should allow for the guard functionality. Thus, when the negative pressure of blood drawn into the inflow lumen 19 causes the catheter 1 to move toward the vessel wall, the apex 31 of the guard 29, rather than the inflow aperture 21, will abut up against the vessel wall.

In this aspect, the difference in height between the apex 31 of the guard portion 29 and the proximal most portion of the inflow aperture 21 helps the guard portion 29 to act as a guard to prevent inflow aperture 21 from contacting or resting against the vessel wall. The exemplified configuration of the guard portion 29 thus functions to ensure that aperture 21 remains positioned away from the vessel wall so as to avoid being partially or completely blocked and compromising outcome of the treatment session. As shown in the figures, the apex 31, with its extended height, provides a separating barrier between the inflow aperture 21 and the outflow aperture 11, which acts to further minimize mixing cleansed and uncleansed blood during a dialysis session and decreasing recirculation problems.

As illustrated along line B-B of FIG. 1B, in one aspect, the guard portion 29 can be configured so that, from a front elevational view, the distal end of the inflow aperture 21 is partially visible, being protected by portions of the apex 31 of the guard portion 29. The outer wall 15 of the distal portion 5 of the catheter 1 transitions into a shared outer wall 18 of the outflow lumen 9 and guidewire lumen 27, which has an inner wall 43. In this aspect, the lumen 27 is surrounded by an expanded guidewire wall segment 100 that separates lumen 27 from outflow lumen 9. Wall segment 100 may be formed using several techniques well known in the art including re-forming existing shaft material, or using a supplemental tip-forming or a molding process. In one aspect, the lumen 27 can be positioned within guidewire wall segment 100 to ensure that the cross-sectional area of outflow lumen 9 at the tapered distal end portion 35 is substantially equivalent to the cross-sectional area of the proximal portion 3 of the lumen 9.

In another aspect, the guidewire lumen 27 shared outer wall 18, combined with the orientation of the tapered distal end portion 35 also protects the outflow aperture 11 from being blocked if the catheter 1 comes into contact with the vessel wall. Referring to FIG. 1A, the catheter shaft 7 may be oriented such that it abuts the vessel wall at distal tip 8 rather than at apex 31 of the guard portion 29. In this orientation, the distal tip 8, with the guidewire exit aperture 39, contacts the vessel wall and provides a spacing function similar to the guard 29 to protect the outflow aperture 21 from contacting and being blocked by the vessel wall. In this exemplified aspect, the tapered distal end portion 35 is angled or oriented away from the vessel wall and will not become occluded by the vessel wall because is it protected by the distal tip 8.

FIG. 2A illustrates a partial enlarged cross-sectional view of the distal portion 5 of the catheter shaft 7 of FIG. 1 with at least a portion of guidewire 20 inserted into the third lumen 27 and an exemplified method of inserting the catheter 1 into a blood vessel and using the catheter 1 to infuse drugs and other solutions into the catheter. In one aspect, the distal portion 5 of the catheter is configured to allow the distal portion 5 of the catheter to be substantially straightened when the guidewire 20 is inserted into and advanced through the third lumen 27 and to allow the substantially curved portion of the distal portion 5 of the catheter to recover toward its unstressed state after the guidewire 20 is removed from the third lumen, as illustrated in FIG. 1A.

In this aspect, the third lumen 27 of the catheter 1 extends substantially through the entire catheter 1 to the proximal portion 3 of the catheter and aids in over-the-wire placement by allowing the distal portion of the catheter 5 to be straightened when at least a portion of guidewire 20 is inserted into the third lumen 27 of the catheter 1. In another aspect, the third lumen 27 is in selective fluid communication with infusates, such as drugs, which allows the third lumen 27 of the catheter 1 to be used for, for example, but not limited to, blood sampling, injection of drug therapies, CT injection, and the like.

As illustrated in FIGS. 2A and 2B, port 10 is in fluid communication with the third lumen 27 and is defined along the catheter shaft 7 proximal to the inflow aperture 21. FIG. 2A illustrates the cross-sectional angled profile of the tapered distal end portion 35 of the catheter 1 with its leading distal tip 8, after the distal portion 5 has been moved into a substantially planar position upon the insertion of a portion of guidewire 20 into the third lumen 27 of the catheter 1. In one aspect, the distal-most leading edge of the tapered end portion 35 terminates in a guidewire exit aperture 39 for optimized guidewire tracking. In a further aspect, the distal end portion 35 also includes a distally-facing, full size outflow lumen 11. Thus, the tapered distal end portion 35 combines the features of a distal end profile capable of tracking over guidewire 20 and dilating the insertion track as well as minimizing vessel wall contact with an aperture that is not reduced in cross-sectional area.

It is contemplated that the distal portion 5 of the catheter 1, with the tapered distal end portion 35, performs several key functions. First, a portion of the tapered distal end portion 35 of the distal portion of the catheter 5 forms an angled leading edge that is configured to facilitate insertion and advancement of the catheter 1. The distally-facing orientation of the tapered distal end portion 35 can be angled away from the vessel wall to minimize engagement with the vessel wall, once inserted. Second, the angled profile of the tapered distal end portion 35 provides an atraumatic dilating function by gradually expanding the tissue track from the approximate size of a guidewire, which is, for example and without limitation, typically 0.035 inches, to the slightly larger diameter of the distal tip 8, to the diameter of the catheter shaft 7 at the proximal most edge of outflow aperture 11, which is, for example and without limitation, approximately 0.160 inches, to the maximum diameter of the catheter shaft 7 at inflow aperture 21, which is, for example and without limitation, approximately 0.203 inches. Thus, the dilating profile of the catheter 1 eliminates the necessity for the use of an introducer sheath.

One skilled in the art will appreciate that the method of inserting the catheter 1 may encompass the use of any of the embodiments of the catheter 1 described herein and illustrated in FIGS. 1 through 7. In one aspect, the method comprises providing the catheter 1 described in any of FIGS. 1 through 7, inserting a guidewire 20 into a vessel in a patient body; inserting the proximal end of the guidewire 20 into the guidewire exit aperture 39 of the guidewire lumen 27; advancing the catheter 1 over the guidewire 20; inserting the catheter 1 into a vessel in a patient body over the guidewire 20; positioning the distal portion of the catheter 1 at a desired location within the target vessel; and removing the guidewire 20 from the catheter.

In another aspect, the method may further comprise straightening the distal portion 5 of the catheter upon insertion of the guidewire 20 into the guidewire lumen 27. In this aspect, and as described above, after the guidewire 20 is inserted into the guidewire lumen 27, the inserted guidewire 20 and the distal portion of the catheter 5 become approximately parallel with the axis of the catheter shaft 7, as illustrated in FIG. 2A.

In a further aspect, when the guidewire 20 is removed from the catheter shaft 7, the distal portion of the catheter 1 is configured to bias back toward and to resume its substantially curved configuration, as illustrated in FIG. 1A. In this aspect, the substantially curved distal portion of the catheter 5 has flexibility and a shaped memory, formed during the manufacturing process of the catheter, which urges the substantially curved distal portion 5 of the catheter to recover to its original curved unstressed state after the guidewire 20 has been removed. Thus, in operation and in one exemplary aspect, the inner angle θ of the guard portion 29 biases back to an angle equal to or greater than about 90 degrees relative to the longitudinal axis of the catheter shaft 7.

In another aspect, when the catheter 1 is deployed in the vessel, the catheter 1 may migrate from the center of the vessel lumen and abut up against a portion of the inner wall of the vessel (not shown). The guard portion 29 is configured to contact the inner vessel wall along a portion of the apex 31 and, as described above, acts as a shield, to prevent the aperture 21 of the catheter from being occluded by contact with the vessel wall. The guard portion 29 also helps to provide a recirculation barrier between the inflow aperture 21 and the outflow aperture 11. In a further aspect, the guard portion 29 can act to orient outflow aperture 11 more centrally within the vessel where blood volume is highest, which can further minimize recirculation rates, increase the efficiency of the dialysis session, and reduce vessel wall trauma caused by sustained contact with the catheter.

One skilled in the art will appreciate that, after the catheter 1 has been inserted in a patient and within days after implantation, fibrin sheaths may grow along the catheter shaft 7 from the vein entry point and progress toward the catheter distal tip 8. In one embodiment, a method of using the inserted catheter 1 to infuse drugs, such as, for example, and without limitation, urokinase or chemotherapeutic drugs, to dissolve the fibrin sheath or other occlusive material on the catheter shaft 7, is provided. Such a method can comprise inserting the vascular access catheter into the patient's body, as described above, providing fluids, such as an infusate, injecting the infusate through the third lumen of the catheter and the port and into the patient's body. In this aspect, the infusate is injected into the third lumen 27 of the catheter 1, the injected infusate travels from the infusion port 4 through extension tube 54 and into the third lumen 27, and exits the at least one port 10 along the catheter shaft 7, where the fibrin sheath growth may be located.

In this aspect, after exiting port 10, the injected infusate flows toward the distal portion of the catheter 5, in the direction of blood flow, where the fibrin sheath may occlude port 10 and/or any distal apertures in the catheter shaft 7, such as, but not limited to apertures 21 and 11. After exiting the port 10, the infusate comes into contact with any fibrin sheath or occlusive material along the catheter shaft 7 and dissolves the occlusive material or fibrin sheath that forms on the catheter shaft 7. Exemplary infusates may comprise, but are not limited to, drugs, anti-restenosis agents, anti-thrombogenic agents, anti-inflammatory agents, anti-thrombotic agents, saline, contrast agents, urokinase, streptokinase, tissue plasminogen activator (t-PA), anti-coagulants, fibrinolytic agents, anti-proliferative agents, chemotherapeutics, and/or the like. This method can more specifically be used to inject a contrast agent under high pressure through the third lumen of the catheter and the port and into the body for computed tomography.

In another aspect, a method of removing occlusive material is provided that involves inserting the catheter described herein into a patient's body, and injecting the infusate through the third lumen of the catheter and the port and into the body, thereby removing any occlusive material that is located on the catheter shaft.

FIG. 2B illustrates two different cross-sectional views of the catheter shaft 7 of the embodiment of FIG. 1A, shown with at least a portion of the guidewire 20 inserted through the lumen 27 of the catheter 1. The first cross-sectional view, D-D, illustrates the substantially double-D lumen configuration of the catheter shaft 7 (with the respective inflow and outflow lumens 19, 9) with a third lumen 27. In one aspect, the substantially double-D lumen configuration extends to where the inflow lumen 19 terminates at aperture 21. Although the lumens of the exemplified catheter 1 have a substantially double-D configuration, it will be appreciated that it is contemplated that the catheter 1 may have any suitable cross-sectional lumen shape as required for the particular use of the catheter 1. It is well known in the art that double-D lumen configurations can allow for maximal flow rates for catheters that have a circular cross-sectional profile.

In one aspect, the outer wall 16 of the catheter shaft 7 surrounds the outflow lumen 9 and the inflow lumen 19, which are separated by the internal septum 17. In this aspect, the outflow lumen 9 extends from the proximal most end of the catheter shaft 7 to aperture 11, which is defined in the tapered distal end portion 35. The inflow lumen 19 extends distally from the proximal most end of catheter shaft 7 to the inflow aperture 21. The outflow lumen 9 has an inner wall 13, and the inflow lumen 19 has an inner wall 25. As illustrated along line D-D, and for example, the internal septum 17 can have a width of approximately 0.144 inches. In this exemplary embodiment, the preferred height of each substantially double-D lumen is approximately 0.064 inches.

In another aspect, the outer wall 16 and inner wall 25 define the inflow lumen 19. The outflow lumen 9 extends distally of the inflow lumen 19, which terminates at inflow aperture 21, proximal to line C-C. At the termination point of inflow aperture 21, the inflow lumen 19 terminates and is continued as a partially single-D lumen 9. In another aspect, the guidewire lumen 27 continues through the entire catheter 1. In this aspect, the inner wall 43 of guidewire lumen 27 is shown in surrounding relationship to guidewire 20 in FIGS. 2A and 2B.

A cross-sectional view of line C-C in the distal portion 5 of the catheter 1 is also illustrated. Along line C-C, the D-shaped outflow lumen has transitioned to a single substantially round outflow lumen 9, and the effective cross-sectional lumen area of the outflow lumen 9 is maintained at its largest diameter to the distal aperture 11. A portion of guidewire 20 is illustrated slidably inserted into guidewire lumen 27, which provides a guidewire track for guidewire 20 to facilitate insertion of the catheter 1 through tissue into the target vessel.

As illustrated along line C-C, the transitional wall 14 represents the inner wall of the internal septum 17 of the outflow lumen 9 at the partial double-D lumen section. In this exemplary example, at line C-C, the outflow lumen 9 has an inner diameter of approximately 0.095 inches and an outer diameter of approximately 0.140 inches. In this example, the rounded outer profile of the catheter shaft 7 at line C-C is of a smaller outer cross-sectional diameter than the cross-sectional diameter of the catheter shaft 7 at line D-D, which measures approximately 0.203 inches. The reduced diameter of the catheter 1 facilitates insertion and advancement of the distal end of the catheter 5 through the tissue track and into the vessel. In a further aspect, the lumen 27 has a substantially circular shape defined by an inner wall 43.

In a further aspect, although the profiles of the lumens 19 and 9 of the catheter 1 change at different sections of the catheter, the effective cross-sectional lumen areas are maintained throughout the length of the catheter 1. Specifically, the effective cross-sectional area of each of the substantially double-D inflow and outflow lumens, taken along line D-D, can exemplarily be approximately 0.0065 inches², which is substantially equal to the cross-sectional area of the outflow catheter 1 taken along line C-C.

In addition, unlike current unitary catheter designs, the catheter 1 allows for insertion over a guidewire utilizing a leading distal end guidewire aperture without increasing the overall diameter of the catheter 1 and without compromising the cross-sectional luminal area of the outflow lumen 9. In one example, the cross-sectional diameter of the tapered distal portion 35 taken along the longitudinal axis of the catheter shaft 7 is about 0.160 inches, but may exemplarily range from between about 0.150 to 0.180 inches.

Accordingly, in one aspect, a catheter 1 with a non-conical tapered dilating distal portion 35 is provided that maintains a substantially consistent, uniform luminal cross-sectional area throughout the entire length of the catheter shaft 7. In this aspect, the substantially open tapered face geometry of the outflow lumen aperture 11 of the distal tip 8 of the catheter allows for maximum blood flow because the cross-sectional area of the outflow lumen 9 is maintained from the proximal portion 3 to the distal portion 5 of the catheter 1, while the outer diameter of the catheter 1 is not effectively increased. Because of its size and orientation, the outflow lumen aperture 11 is not likely to occlude, compared with conventional conical-tapered or blunt tip catheters with smaller side wall lumen openings.

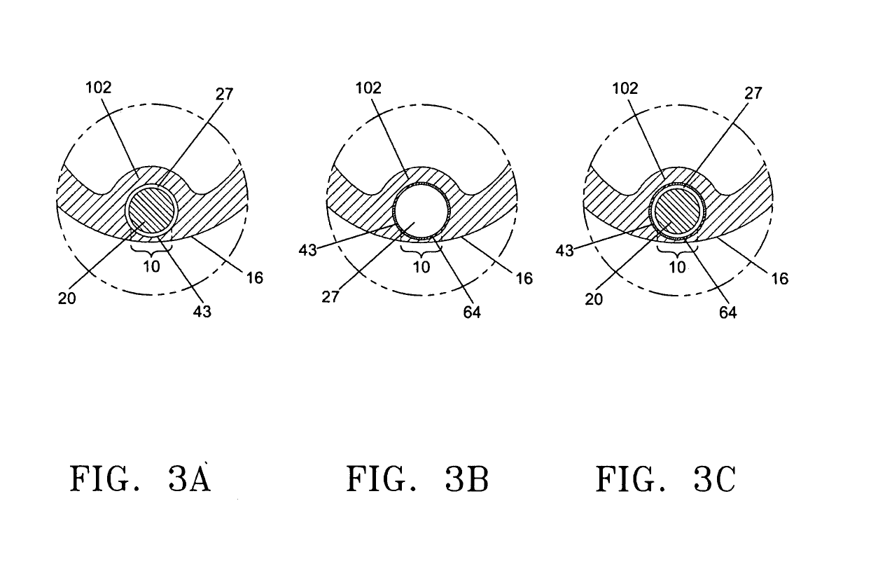

FIG. 3A illustrates a partial exploded cross-sectional view of the third lumen 27 of the catheter with the inner wall 43 of the lumen 27 positioned in surrounding relationship to guidewire 20 when a portion of the guidewire 20 is inserted into the guidewire lumen 27. Port 10 is shown exiting through the third lumen 27 at the bottom of the catheter shaft 7.

In an alternative embodiment, and as illustrated in FIGS. 3B and 3C, the guidewire lumen 27 may have a liner 64 that is positioned on at least a portion of the inner wall 43 of the lumen 27 to form a reinforced lumen 27 that is configured to be in surrounding relationship to the guidewire 20 that is inserted into the lumen 27. In this aspect, the liner 64 can be a tubular structure that functions to increase the burst pressure of the guidewire lumen 27. Burst pressure is defined herein as the amount of pressure that the lumen 27 may withstand during high pressure applications, such as contrast media injections, before rupturing. In this aspect, the liner 64 can be formed of a liner material with a higher yield stress than the material of the catheter shaft 7.

In various aspects, the liner 64 can protect the inner wall 43 of the lumen 27 from erosion due to drug and chemical use, thereby allowing the catheter 1 to be more resistant to drug therapy, and also supports high pressures for the purpose of CT injection, thereby allowing the catheter to be effectively used for high pressure CT injection, and eliminating the need for port placement. As noted above, the liner may be made of any suitable material that may increase the burst pressure of the lumen 27, such as, but not limited to, nylon, polyamide, and the like. The liner 64 can also function to reduce friction over the guidewire 20, which enhances the guidewire tracking capabilities of the lumen 27. In one example, the liner 64 may have a wall thickness of between approximately 0.002 and 0.005 inches. Optionally, the liner 64 can comprise a higher strength material than the catheter shaft 7 to allow for the formation of thinner surrounding catheter wall sections 102, thereby minimizing reduction in luminal cross-sectional area of the inflow 19 and outflow 9 lumens.

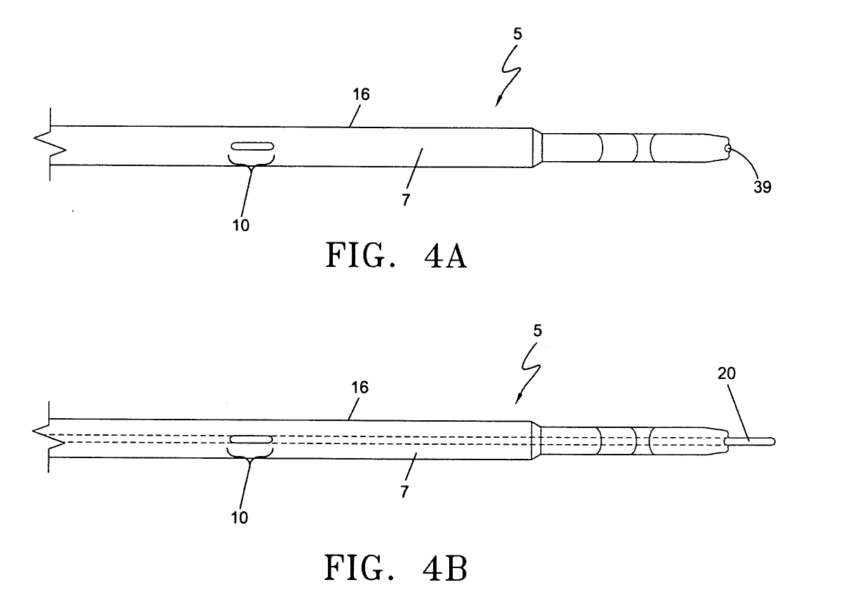

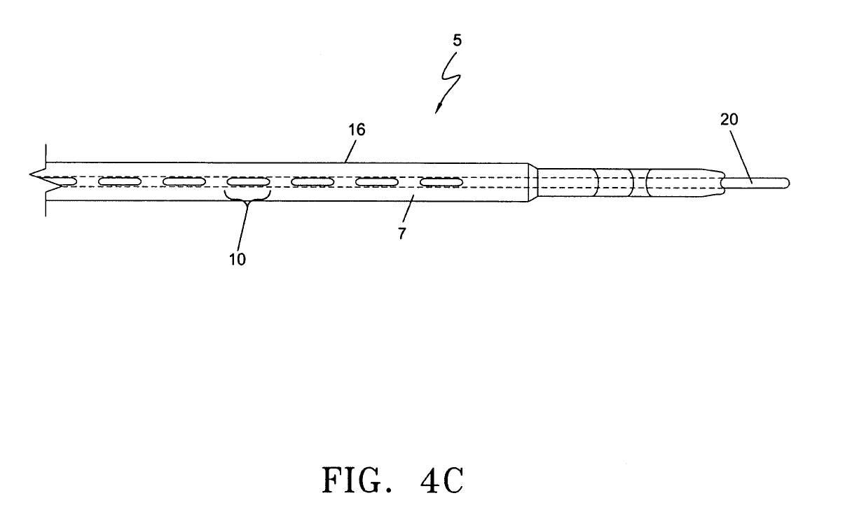

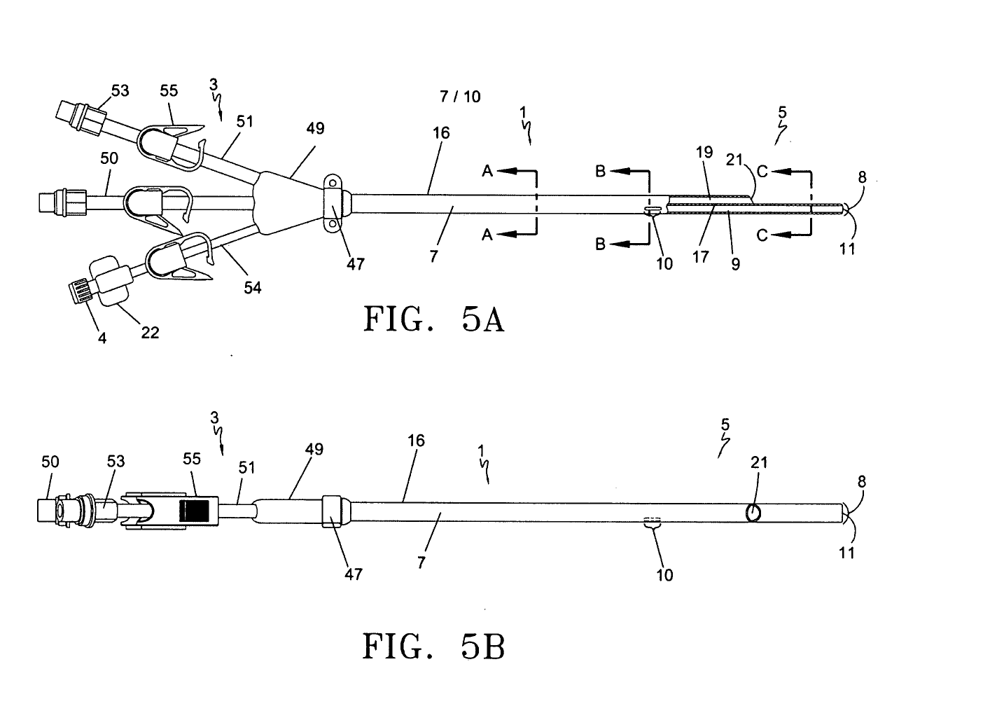

FIG. 4A illustrates a partial bottom view of the distal end portion of the catheter shaft 7 of FIGS. 1A through 3C. The catheter shaft 7 has at least one port 10, positioned proximally of the inflow lumen 21, that is in fluid communication with the third lumen 27 therein the catheter shaft 7. In one exemplary aspect, the port 10 can be positioned proximally between approximately 5 cm and 7 cm from the distal tip 8 of the catheter and on the bottom side of the catheter shaft 7. In one aspect, the proximal port 10 is sized to be smaller in diameter than the guidewire 20, so that as the guidewire 20 is advanced through the guidewire lumen 27, the guidewire does not exit out of the port 10. In one exemplary aspect, the width of the port 10 can be approximately equal to or smaller than approximately 0.035 inches in diameter. For example, the port 10 may be, but is not limited to, a diameter of between approximately 0.032 inches and 0.035 inches. Further, the length of the port 10 can be exemplarily between approximately 1 mm and 5 mm. Optionally, and in one aspect, the port 10 may be of any suitable dimension, provided that the width of the port 10 is not greater than the width of the guidewire 20 so as to prevent the guidewire 20 from exiting the port 10 when the guidewire 20 is inserted into and through the third lumen 27.

As noted above, the exemplified position of the port 10 helps to deliver drugs, such as urokinase, through the port 10, to dissolve fibrin sheaths or other occlusive material that have the tendency to form along the catheter shaft 7. In this aspect, the exemplified placement of the port 10 along the catheter shaft 7 spaced from and proximal from the distal tip 8 allows fibrin sheath-dissolving or anti-thrombolytic drugs to be delivered through the port 10 directly into or in the general vicinity of the fibrin sheath itself that may form along the catheter shaft 7, in contrast to current triple lumen dialysis catheters. This allows the drugs to dissolve fibrin sheaths or other occlusive material more proximally of the distal tip 8 and the inflow aperture 21 of the catheter, instead of solely at the distal tip 8 of the catheter, as is often described in the prior art.

Because it has been theorized that fibrin sheaths tend to grow from the venous entry point down the catheter shaft to the distal tip, the exemplified placement of the port 10 along the catheter shaft 7 spaced from and proximal from the distal tip 8 overcomes problems with current dialysis catheters that have drug infusion lumens with apertures limited to just the distal tip of the catheter or the general vicinity of the distal tip of the catheter. In such prior art dialysis catheters, the infusion of drugs at the distal tip of the catheter is not effective for dissolving fibrin sheaths or other occlusive material that grow along the catheter shaft 7 because the drugs are usually infused at the distal end of the catheter, and the drugs get washed away with the blood flow, thereby rendering such drugs virtually useless for dissolving fibrin sheath growth.

The catheter 1 is also more cost-effective, saves time, and causes less trauma to the patient, compared to prior art dialysis catheters because the catheter design negates the need to replace the dialysis catheter due to fibrin growth. The catheter allows for the effective elimination of undesirable fibrin sheath growth by allowing for the infusion of drugs directly into the fibrin sheath from within the catheter, instead of having to replace the dialysis catheter due to fibrin growth or attempting to infuse the drug at the very distal tip of the catheter. It is contemplated that, although the proximal port 10 is disclosed herein as part of a dialysis catheter, the proximal port 10 feature described herein may also be incorporated into any triple lumen acute or chronic catheter, such as, but not limited to peripherally inserted central catheters (PICCs), port catheters, central venous catheters (CVCs), or other types of catheters.

FIG. 4B illustrates a partial bottom view of the distal end of the catheter shaft 7 of FIGS. 1A through 3C with a portion of guidewire 20 inserted into the third lumen 27. FIG. 4C illustrates a partial bottom view of an additional embodiment of the catheter shaft 7 in which a plurality of ports 10 are defined in and are spaced along the catheter shaft 7, proximal to the inflow lumen aperture 21. In one aspect, such ports 10 may be equi-distant from one another. Optionally, each port 10 may be spaced from an adjacent port 10 at different distances along the catheter shaft 7. In a further aspect, the plurality of ports 10 may be positioned on a plane that substantially bisects the longitudinal axis of the catheter shaft 7. It is contemplated that the ports 10 may be of the same size or shape, or they may be of different sizes or shapes. The placement of the plurality of ports 10 in fluid communication with the third lumen 27 of the catheter shaft allows for greater and longer distributions of drugs to break up fibrin sheath growth, and for enhanced blood sampling, as compared to a sole proximal port 10, which allows for enhanced CT injection capabilities. The plurality of ports 10 may also help to reduce potential whipping of the catheter shaft 7 due to the availability of additional pressure exits points through the port 10, and may eliminate the need to reposition the catheter 1 during drug infusion.

As described above, anti-thrombotic drugs, such as urokinase, or any other type of suitable drug or infusate solution, may be injected into the infusion port 4, and from there, infused through the extension tube 54 and into the lumen 27 of catheter 1 under appropriate pressure so that the drugs will exit the lumen 27 through port 10 in order to come into contact with fibrin build up, causing the fibrin to dissolve and be removed. Drugs such as urokinase can typically be administered in doses ranging from 5,000 units to 250,000 units over 30 minutes to 3 hours. This exemplified dosage will cause the fibrin sheath to break up, solubilize and be carried away with the blood flow in the vascular system surrounding the catheter 1. The resulting fibrin sheath removal will decrease the chance of bacterial growth, and the inflow and outflow lumen apertures 19 and 9, respectively, will be unclogged of any build up of fibrin. Optionally, if the fibrin sheath has developed over the port 10, the infusion of lytic drugs through the port 10 and into the overlying fibrin sheath helps to unclog the port 10, which then allows the infusion of drugs to treat the fibrin sheath or other occlusive material. Allowing contact of anti-thrombotic agents with the fibrin sheath or other occlusive material is most important in getting rid of fibrin sheaths or other occlusive material that can build up along a catheter shaft 7.

It is contemplated that the force of injected drugs or other fluids under appropriate pressure into the lumen 27 and exiting the port 10 may cause the catheter 1 to radially expand, such that it may mechanically disrupt and break up the fibrin sheath on the outside surface of the catheter shaft 7. The port 10 in the catheter 1 can thus be used to quickly and easily remove any fibrin sheaths which may be formed on the outside surface of the catheter shaft 7 and which may have been mechanically disrupted by the force of the drug injection. This eliminates the need to replace the catheter 1 or mechanically strip the fibrin sheath from the outside surface of the catheter shaft 7. Thus, in one aspect, the catheter 1 provides a means to eliminate the sudden release of fibrotic material that may travel to the patient's lungs, which often occurs during mechanical stripping procedures. The design of the catheter 1, in various aspects, also eliminates the need to create an additional entry site in the patient's body to insert snares or other mechanical devices during mechanical stripping procedures. In further aspects, the use of the catheter 1 also eliminates possible damage to the catheter 1, which extends its useful life, and reduces or eliminates the possibility of bacterial infection, which could potentially occur if the fibrin sheath remains on the catheter shaft 7.

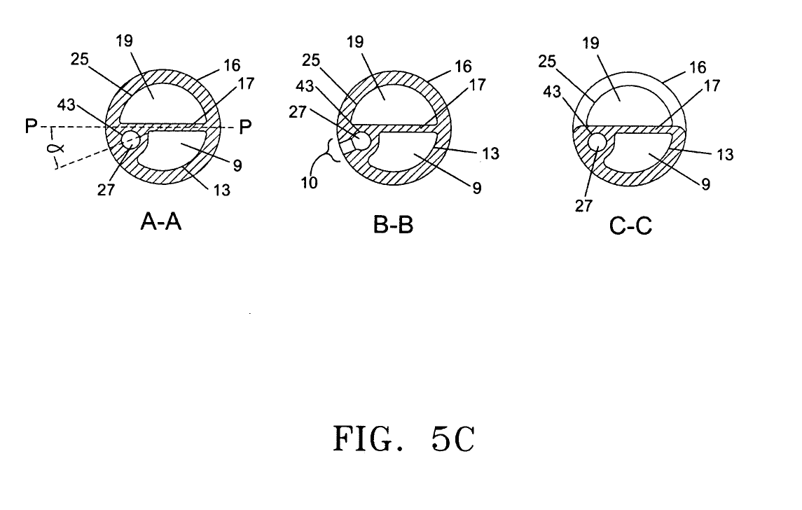

FIG. 5A illustrates a plan view of an additional embodiment of the vascular access catheter 1. In this aspect, the catheter 1 comprises a proximal portion 3 and a distal portion 5. In this embodiment, the catheter 1 has a distal portion 5, which is substantially straight and does not have an angled edge. In one aspect, the distal tip 8 of the catheter can have slightly rounded edges. Further, as described above, the catheter shaft 7 can be comprised of an outer wall 16 and at least two longitudinal lumens 19 and 9 that extend longitudinally substantially the entire length of the catheter shaft 7. The lumen 19 is in fluid communication with the extension tube 51, and the lumen 9 is in fluid communication with the extension tube 50. Both extension tubes 50, 51 communicate through the bifurcate 49.

It is contemplated that the dimensions and materials of the catheter 1 illustrated in FIGS. 5A through 7 may be identical or similar to the materials and dimensions of the catheter described in the previous embodiment. The distal portion of the catheter of this embodiment is flexible, but it is not manufactured with a shape memory, in contrast to the embodiment described in FIGS. 1A-4C. As described in the previous embodiment in FIGS. 1A and 1B, the catheter 1 can have a proximal port 10. In one aspect, the port 10 of the catheter 1 described in FIGS. 5A and 5B can have a port 10 that is defined in the exterior surface of the catheter shaft 7 substantially transverse to a plane bisecting the internal septum 17. In another aspect, the port 10 is defined in the exterior surface of the catheter shaft 7 at an acute angle α relative to a plane bisecting the internal septum 17 of the catheter 1 that is shared by the lumens 19 and 9, instead of exiting along the bottom of the catheter shaft 7 below the outflow lumen 9 as in the previous embodiment described in FIGS. 1A and 1B.

FIG. 5B illustrates a top plan view of the catheter 1 of FIG. 5A in which the inflow aperture 21 of the inflow lumen 19 is exemplarily defined approximately 2.5 cm from the distal most edge of the guidewire exit aperture 39. It is contemplated that the inflow aperture 21 may be positioned any suitable distance from the distal tip of the catheter 8. The inflow aperture 21 is distally-facing, and can be positioned at an angle that is greater than about 90 degrees proximally from and relative to the longitudinal axis of the catheter shaft 7. In one example, the port 10 can be positioned approximately 5 cm to 7 cm from the distal tip 8 of the catheter 1 along the catheter shaft 7 and can be defined in the side of the catheter shaft 7 at an angle α of approximately 22 degrees relative to a plane bisecting the internal septum 17 of the catheter shaft 7. Alternatively, the port 10 may exit the catheter shaft 7 at any suitable angle relative to a plane bisecting the internal septum 17 of the catheter shaft 7.

FIG. 5C illustrates three cross-sectional views of the catheter of FIG. 5A, along lines A-A, B-B, and C-C, respectively. In the cross-section taken along line A-A of the catheter shaft 7, the catheter 1 is surrounded by outer wall 16, and has an inflow lumen 19 with an inner wall 25, an outflow lumen 9 with an inner wall 13, and a third guidewire lumen 27 with an inner wall 43 that is defined within the catheter shaft 7 adjacent to and on the same side of the internal septum as the outflow lumen 9. As described in the embodiment depicted in FIGS. 3A, 3B, and 3C, the third lumen 27 may allow the infusion of certain anti-thrombotic drugs and may also comprise a reinforced liner 64 placed inside of the third lumen to provide resistance to chemicals and to allow high pressure CT injections. In one aspect, the outflow lumen 9 is partially D-shaped on one side and can be configured to curve inward on the other side to allow space for the third lumen 27 that is positioned adjacent to the inflow lumen 9.

In this embodiment, the outflow lumen 9 is substantially smaller in transverse cross-sectional area than the inflow lumen 19. In one example, the inflow lumen 19 may have a transverse cross sectional area of approximately 0.0065 inches², and the outflow lumen 9 may have a transverse cross-sectional area of 0.0043 inches². The inflow lumen 19 and outflow lumen 9 are separated by an internal septum 17. In one aspect, the diameter of the inner wall 43 of the guidewire lumen 27 is substantially smaller than inflow lumen 19 and the outflow lumen 9 and is configured to allow for infusion of drugs and guidewire tracking.

The cross-section taken along line B-B illustrates port 10 positioned in the third lumen 27 along the catheter shaft 7. The port 10 is defined in the catheter shaft 7 at a downward angle of approximately 22 degrees relative to a plane bisecting the internal septum 17, which forms at least part of the third lumen 27. One skilled in the art would appreciate that drugs may be infused through the port 10 to dissolve fibrin sheath buildup along the catheter shaft 7. In a further aspect, the diameter of the port 10 can be smaller than the guidewire 20, such as, for example, the port 10 may have a diameter of approximately 0.024 inches. However, it is contemplated that the port 10 may have any suitable diameter, as long as it is smaller in diameter than the diameter of the guidewire 20, so as to prevent the guidewire 20 from exiting the catheter 1 via the port 10. The cross-section along line C-C illustrates the inflow lumen 19, the outflow lumen 9, and the third lumen 27 of the catheter 1. As described in the previous embodiment, the inflow lumen aperture 21 terminates proximally of the outflow lumen aperture 11.

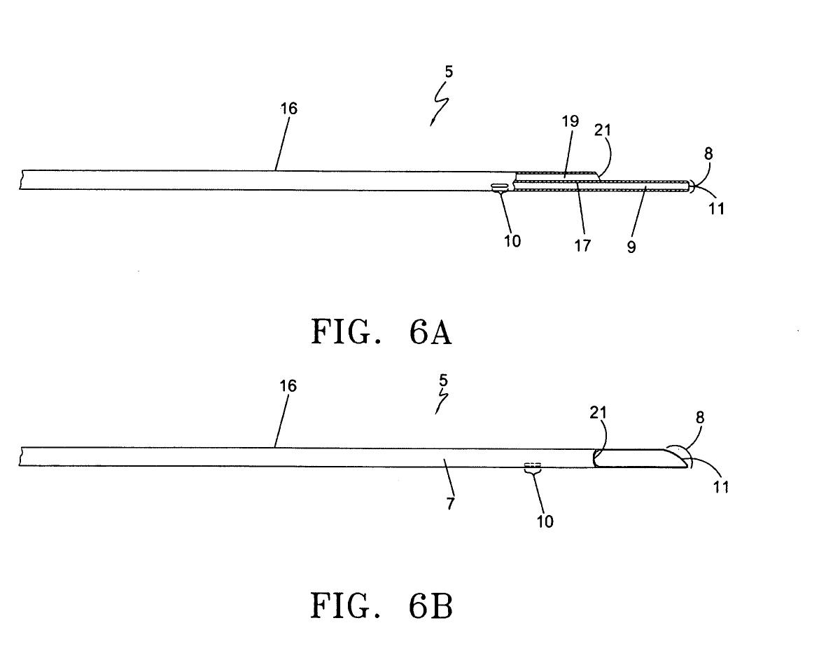

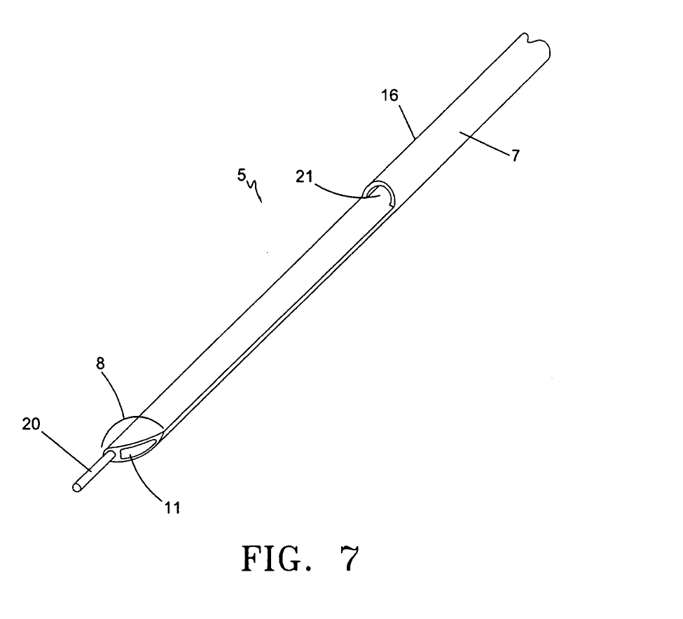

FIG. 6A illustrates a partial cross-sectional side view of an additional embodiment of the catheter 1 in which the distal tip of the catheter 8 has an angled leading distal edge or tip 8 (not shown), and a proximal port 10 that is in fluid communication with the third guidewire lumen 27 and is defined in the side of the catheter shaft 7, as described in the embodiment in FIGS. 5A, 5B, and 5C above. The respective inflow lumen 19 and outflow lumen 9, separated by the internal septum 17 are illustrated as well as their respective inflow and outflow apertures 21, 11. FIG. 6B illustrates a top plan view of the catheter of FIG. 6A, with an angled leading distal tip or edge 8 and without a guidewire 20 exiting the distal tip 8 of the catheter. This embodiment of the catheter 1 provides an atraumatic leading edge for catheter tunneling during insertion and tunneling of the catheter along a tissue track inside a vessel.

FIG. 7 illustrates a perspective view of the catheter 1 in which the angled leading edge 8 is illustrated with a portion of guidewire 20 inserted into guidewire lumen 27 and exiting the guidewire lumen 27 of the catheter 1. In one aspect, the angled leading edge 8 of the illustrated catheter is less traumatic compared to blunt, open ended catheters, which may be appreciably more difficult to insert into the patient. In this aspect, the angled leading edge 8 acts as a dilator and allows the dilating tip to be easily advanced through a vessel track and into the vessel. The inflow lumen aperture 21 and outflow lumen aperture 11 are also visible in this perspective view.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein, which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the selected embodiments of the invention. Those skilled in the art may recognize other equivalents to the specific embodiments described herein which equivalents are intended to be encompassed by the claims attached hereto.

What is claimed is:

1. A vascular access catheter, comprising:
a catheter shaft, wherein at least a portion of the catheter shaft forms a distal portion of the catheter, and wherein the distal portion has a distal end portion having a distal tip, wherein at least a portion of the distal end portion has an angled edge that is angled proximally away from the distal tip and is positioned at an acute angle relative to a longitudinal axis of the catheter shaft, the catheter shaft further comprising:
a plurality of longitudinally extending lumens defined therein the catheter shaft, wherein the plurality of lumens comprises a first lumen, a second lumen, and a third lumen, wherein the first and third lumens extend substantially the entire length of the catheter shaft, and wherein the third lumen is configured to selectively receive at least a portion of a guidewire;
a first lumen aperture defined in at least a portion of the distal end portion that has an angled edge, adjacent the distal tip and in fluid communication with the first lumen;
a second lumen aperture defined therein the exterior surface of the catheter shaft and in fluid communication with the second lumen, wherein the second lumen aperture is spaced proximally from the first lumen aperture; and
a port defined therein the catheter shaft and in fluid communication with the third lumen and the exterior surface of the catheter shaft, wherein the port is spaced proximally from the second lumen aperture.

2. The catheter of claim 1, wherein the third lumen has a distal aperture that is defined therein the distal portion at the distal most portion of the angled edge.

3. The catheter of claim 1, wherein the cross-sectional area of the first lumen is substantially uniform throughout the entire length of the catheter.

4. The catheter of claim 1, wherein second lumen aperture is positioned at an angle greater than about 90 degrees relative to the longitudinal axis of the catheter shaft.

5. The catheter of claim 1, wherein at least a portion of the distal end portion is tapered, and wherein the third lumen aperture is defined therein the distal most portion of the angled edge of the distal end portion, distal of the first lumen aperture.

6. The catheter of claim 1, wherein the acute angle of the angled edge of the at least a portion of the distal end portion is between about 15 degrees and 75 degrees relative to the longitudinal axis of the catheter shaft.

7. The catheter of claim 6, wherein the acute angle of the angled edge of the at least a portion of the distal end portion is approximately 30 degrees relative to the longitudinal axis of the catheter shaft.

8. The catheter of claim 1, wherein the distal portion of the catheter is substantially straight.

9. The catheter of claim 1, wherein at least a portion of the distal portion of the catheter is substantially curved in an unstressed state.

10. The catheter of claim 9, wherein, in the unstressed state, the at least a portion of the distal portion of the catheter is angled at an inner angle between about 45 degrees and 135 degrees.

11. The catheter of claim 10, wherein the inner angle is approximately 90 degrees.

12. The catheter of claim 9, wherein at least a portion of the distal portion of the catheter that is substantially curved comprises a guard portion.

13. The catheter of claim 12, wherein a portion of the guard portion is spaced from the longitudinal axis of the catheter shaft a distance equal to or greater than the distance an outer wall of the second lumen aperture is spaced from the longitudinal axis of the catheter shaft.

14. The catheter of claim 9, wherein the distal portion of the catheter is configured to allow the distal portion of the catheter to be substantially straightened when the guidewire is inserted into and advanced through the third lumen and to allow the substantially curved portion of the distal portion of the catheter to recover toward its unstressed state after the guidewire is removed from the third lumen.

15. The catheter of claim 1, further comprising a liner, wherein the liner is positioned thereon at least a portion of an inner wall of the third lumen.

16. The catheter of claim 1, wherein the catheter comprises a plurality of ports that are positioned proximal to the second lumen aperture, wherein each port of the plurality of ports is spaced from an adjacent port.

17. The catheter of claim 16, wherein the plurality of ports are positioned along a plane that bisects the longitudinal axis of the catheter shaft.

18. The catheter of claim 1, wherein the first and second lumens share a common internal septum, and wherein the port has a port aperture that is defined in the exterior surface of the catheter shaft substantially transverse to a plane bisecting the internal septum.

19. The catheter of claim 1, wherein the first and second lumens share a common internal septum, and wherein the port has a port aperture that is defined in the exterior surface of the catheter shaft at an acute angle relative to a plane bisecting the internal septum.

20. The catheter of claim 1, wherein the first and second lumens share a common internal septum, and wherein the transverse cross-sectional areas of the first and second lumens are substantially equal.

21. The catheter of claim 1, wherein the first lumen has a smaller transverse cross-sectional area than the second lumen.

22. The catheter of claim 1, wherein the third lumen is in selective fluid communication with an infusate.

23. The catheter of claim 22, wherein the infusate is selected from the group consisting of anti-restenosis agents, anti-thrombogenic agents, anti-inflammatory agents, anti-thrombotic agents, saline, contrast agents, urokinase, streptokinase, tissue plasminogen activator (t-PA), anti-coagulants, fibrinolytic agents, anti-proliferative agents, or chemotherapeutics.

24. The catheter of claim 1, wherein the catheter is a hemodialysis catheter.

25. A vascular access catheter, comprising:
a catheter shaft, wherein at least a portion of the catheter shaft forms a distal portion of the catheter, and wherein the distal portion has a distal end portion having a distal tip, the catheter shaft further comprising:
a plurality of longitudinally extending lumens defined therein the catheter shaft, wherein the plurality of lumens comprises a first lumen, a second lumen, and a third lumen, wherein the first and third lumens extend substantially the entire length of the catheter shaft and are separated and share a common internal septum, wherein the first lumen has a smaller transverse cross-sectional area than the second lumen, and wherein the third lumen is configured to selectively receive at least a portion of the guidewire;
a first lumen aperture defined therein the distal end portion of the catheter adjacent the distal tip and in fluid communication with the first lumen;
a second lumen aperture defined therein the exterior surface of the catheter shaft and in fluid communication with the second lumen, wherein the second lumen aperture is spaced proximally from the first lumen aperture; and
a port defined therein the catheter shaft and in fluid communication with the third lumen and the exterior surface of the catheter shaft, wherein the port is spaced proximally from the second lumen aperture.

26. The catheter of claim 25, further comprising a liner, wherein the liner is positioned thereon at least a portion of an inner wall of the third lumen.

27. The catheter of claim 25, wherein the transverse cross-sectional area of each of the plurality of lumens is substantially constant along the respective lengths of the lumens.

28. The catheter of claim 25, wherein the third lumen has a smaller transverse cross-sectional area than the first lumen.

29. The catheter of claim 25, wherein the port has a port aperture that is defined in the exterior surface of the catheter shaft at an acute angle relative to a plane bisecting the internal septum.

30. The catheter of claim 25, wherein the third lumen is in selective fluid communication with an infusate.

31. The catheter of claim 30, wherein the infusate is selected from the group consisting of anti-restenosis agents, anti-thrombogenic agents, anti-inflammatory agents, anti-thrombotic agents, saline, contrast agents, urokinase, streptokinase, tissue plasminogen activator (t-PA), anti-coagulants, fibrinolytic agents, anti-proliferative agents, or chemotherapeutics.

32. A method of inserting a vascular access catheter, wherein the method comprises:
a. providing a vascular access catheter, wherein the catheter comprises a catheter shaft, wherein at least a portion of the catheter shaft forms a distal portion of the catheter, and wherein the distal portion has a distal end portion having a distal tip, wherein at least a portion of the distal end portion has an angled edge that is angled proximally away from the distal tip and is positioned at an acute angle relative to a longitudinal axis of the catheter shaft, the catheter shaft further comprising:
a plurality of longitudinally extending lumens defined therein the catheter shaft, wherein the plurality of lumens comprises a first lumen, a second lumen, and a third lumen, wherein the first and third lumens extend substantially the entire length of the catheter shaft, and wherein the third lumen is configured to selectively receive at least a portion of a guidewire;
a first lumen aperture defined in at least a portion of the distal end portion that has an angled edge, adjacent the distal tip and in fluid communication with the first lumen;
a second lumen aperture defined therein the exterior surface of the catheter shaft and in fluid communication with the second lumen, wherein the second lumen aperture is spaced proximally from the first lumen aperture; and
a port defined therein the catheter shaft and in fluid communication with the third lumen and the exterior surface of the catheter shaft, wherein the port is spaced proximally from the second lumen aperture;
b. inserting the catheter into a vessel in a patient body over the guidewire;
c. positioning the distal portion of the catheter at a desired location within the target vessel; and
d. removing the guidewire from the third lumen.

33. A method of infusing an infusate into a patient body, wherein the method comprises:
a. providing a vascular access catheter, wherein the vascular access catheter comprises a catheter shaft, wherein at least a portion of the catheter shaft forms a distal portion of the catheter, and wherein the distal portion has a distal end portion having a distal tip, wherein at least a portion of the distal end portion has an angled edge that is angled proximally away from the distal tip and is positioned at an acute angle relative to a longitudinal axis of the catheter shaft, the catheter shaft further comprising:
a plurality of longitudinally extending lumens defined therein the catheter shaft, wherein the plurality of lumens comprises a first lumen, a second lumen, and a third lumen, wherein the first and third lumens extend substantially the entire length of the catheter shaft, and wherein the third lumen is configured to selectively receive at least a portion of a guidewire;
a first lumen aperture defined in at least a portion of the distal end portion that has an angled edge, adjacent the distal tip and in fluid communication with the first lumen;
a second lumen aperture defined therein the exterior surface of the catheter shaft and in fluid communication with the second lumen, wherein the second lumen aperture is spaced proximally from the first lumen aperture; and
a port defined therein the catheter shaft and in fluid communication with the third lumen and the exterior surface of the catheter shaft, wherein the port is spaced proximally from the second lumen aperture;
b. inserting the vascular access catheter into the patients body;
c. providing an infusate;
d. injecting the infusate through the third lumen of the catheter and the port and into the body.

34. The method of claim 33, wherein the infusate is selected from the group consisting of anti-restenosis agents, anti-thrombogenic agents, anti-inflammatory agents, anti-thrombotic agents, saline, contrast agents, urokinase, streptokinase, tissue plasminogen activator (t-PA), anti-coagulants, fibrinolytic agents, anti-proliferative agents, or chemotherapeutics.

35. The catheter of claim 33, wherein the third lumen has a distal aperture that is defined therein the distal portion of the catheter shaft at the distal most portion of the angled edge.

36. The method of claim 33, wherein the method further comprises injecting a contrast agent under high pressure through the third lumen of the catheter and the port and into the body for computed tomography.

37. The method of claim 32, wherein the catheter is a hemodialysis catheter.

38. A method of removing occlusive material, wherein the method comprises:
a. providing a vascular access catheter, wherein the vascular access catheter comprises a catheter shaft, wherein at least a portion of the catheter shaft forms a distal portion of the catheter, and wherein the distal portion has a distal end portion having a distal tip, wherein at least a portion of the distal end portion has an angled edge that is angled proximally away from the distal tip and is positioned at an acute angle relative to a longitudinal axis of the catheter shaft, the catheter shaft further comprising:
a plurality of longitudinally extending lumens defined therein the catheter shaft, wherein the plurality of lumens comprises a first lumen, a second lumen, and a third lumen, wherein the first and third lumens extend substantially the entire length of the catheter shaft, and wherein the third lumen is configured to selectively receive at least a portion of a guidewire;
a first lumen aperture defined in at least a portion of the distal end portion that has an angled edge, adjacent the distal tip and in fluid communication with the first lumen;
a second lumen aperture defined therein the exterior surface of the catheter shaft and in fluid communication with the second lumen, wherein the second lumen aperture is spaced proximally from the first lumen aperture; and
a port defined therein the catheter shaft and in fluid communication with the third lumen and the exterior surface of the catheter shaft, wherein the port is spaced proximally from the second lumen aperture;
b. inserting the vascular access catheter into the patient's body;
c. providing an infusate;
d. injecting the infusate through the third lumen of the catheter and the port and into the body, thereby removing any occlusive material that is located on the catheter shaft.

39. The method of claim 38, wherein the infusate is selected from the group consisting of anti-restenosis agents, anti-thrombogenic agents, anti-inflammatory agents, anti-thrombotic agents, saline, contrast agents, urokinase, streptokinase, tissue plasminogen activator (t-PA), anti-coagulants, fibrinolytic agents, anti-proliferative agents, or chemotherapeutics.

40. The catheter of claim 38, wherein the third lumen has a distal aperture that is defined therein the distal portion of the catheter shaft at the distal most portion of the angled edge.

41. The method of claim 38, wherein the catheter is a hemodialysis catheter.
